(19) United States
(12) Patent Application Publication
(10) Pub. No.: US 2003/0139632 A1
(43) Pub. Date: Jul. 24, 2003

(54) Process for producing ether

(76) Inventors: Outi Krause, Espoo (FI); Reetta Karinen, Espoo (FI); Juhani Aittamaa, Helsinki (FI)

Correspondence Address: BIRCH STEWART KOLASCH & BIRCH, PO BOX 747, FALLS CHURCH, VA, 22040-0747, US

(21) Appl. No.: 10/257,272

(22) PCT Filed: Apr. 9, 2001
(86) PCT No.: PCT/FI01/00351

(51) Int. Cl. C07C 41/06

(52) U.S. Cl. 568/689

(57) ABSTRACT

This invention concerns a process and an apparatus for producing ether and the use of a dimerised reaction product as the olefinic feedstock in etherification. According to the present process alcohol and C₈-C₁₂ olefin(s) are fed to a first reaction zone (21-25) comprising at least one reactor, the olefin(s) and alcohol are subjected to an etherification reaction in the presence of an acidic catalyst. The effluent from the first reaction zone (21-25) is fed to a fractionation zone (26). From the fractionation zone (26) is withdrawn a side-flow which is conducted either back to the first reaction zone (21-25) or to a second reaction zone. The effluent of the first reaction zone (21-25) or the second reaction zone is circulated back to the fractionation zone (26), and the ether is recovered from the fractionation zone (26).

### BACKGROUND OF THE INVENTION

1. Field of the Invention

The present invention concerns an apparatus and a process for producing ether. In particular, the invention relates to a process in which dimerised olefinic hydrocarbon feed is etherified to form an ether exhibiting a high octane number. Further, the present invention concerns the use of the effluent of a dimerisation process as feedstock for etherification.

2. Description of Related Art

The octane number of the automotive fuels is increased by adding components with a high octane number to it. An example of such a component is methyl-tert-butyl ether, MTBE. Alternative components are C₄-alkylate and isomerates. The alkylate is typically produced by alkylating isobutane and n-butene, whereby trimethyl pentanes and dimethyl hexanes are obtained. C₅-fraction has previously been used for producing ethers, such as tert-amyl methyl ether, TAME or tert-amyl ethyl ether, TAEE. Both these ethers have been used together with or instead of MTBE to increase the octane number of the automotive fuels. More recently, also synthesised isooctane (2,2,4-trimethylpentane) has been suggested to find use as a fuel component. The octane numbers (Research Octane Number, RON and Motor Octane Number, MON) of isooctane are by definition 100.

It is known in the art that oxygen-containing molecules, such as methanol, MTBE, tert-butyl alcohol (TBA) and water increase the dimer selectivity and thus decrease the selectivity of the trimerising or tetramerising reactions when dimerising olefins in the presence of an ion-exchange resin catalyst. In that connection, we refer to what is stated in U.S. Pat. Nos. 4,375,576 and 4,100,220.

U.S. Pat. No. 4,080,180 suggests that methyl-1,1,3,3-tetramethylbutyl ether can also be used as a fuel component. The ether can be prepared from diisobutene and methanol using acidic catalysts. The process for producing said ether comprises refluxing diisobutene with methanol in the presence of, for example, ion-exchange catalyst for 12 to 24 hours. The publication does not, however, disclose how the diisobutene is produced. GB application 2 325 237 discloses a process for selective dimerisation of isobutene, in which primary alcohol and alkyl ether are fed to the process together with isobutene-containing hydrocarbon feed. The molar ratio of alcohol to isobutene is less than 0.2 in the feed. The molar ratio of alcohol and alkyl ether together to isobutene in the feed is more than 0.1. It is, however, stated in the publication that the best range of the latter molar ratio actually varies from between 0.2 and 0.6 to between 0.3 and 0.6 and between 0.5 and 0.7 depending on the composition of the hydrocarbon feed. Thus, the molar ratio in the feed is kept relatively small. The publication states that the obtained mixture can be conducted to hydrogenation.

### SUMMARY OF THE INVENTION

It is an aim of the present invention is to eliminate the limitations of prior art and to provide a novel process and apparatus for etherifying C₈-C₁₂ olefins and a novel process for producing ether from an olefinic hydrocarbon feed.

It is a further aim of the present invention to provide a novel use for the dimerised reaction product obtained from dimerisation.

The present invention is based on the finding that by etherifying C₈-C₁₂ olefin or a mixture of C₈-C₁₂ olefins with an alcohol or a mixture of alcohols in an etherification zone comprising at least one reaction zone and at least one fractionation zone, is obtained ether(s) having a high octane number. The olefin(s) are fed to a first reaction zone in which they are reacted with alcohol(s) in the presence of a catalyst. The effluent of the first reaction zone is fed to the fractionation zone. A side-flow is withdrawn from the fractionation zone and circulated either back to the first reaction zone or to a second reaction zone. The effluent of the reaction zone is then fed back to the fractionation zone. The ether is recovered from the fractionation zone.

According to a preferred embodiment of the invention, the ether having a high octane number can be produced from an olefinic hydrocarbon feedstock by dimerising at least part of the olefins and etherifying at least part of the dimerised olefins. The dimerisation is carried out in the presence of an oxygen-containing compound, which is then separated in a distillation zone from the effluent of the dimerisation zone and circulated back to dimerisation.

More specifically, the present process for producing ether is characterised by what is stated in the characterising part of claim 1.

The present process for etherification of olefin(s) is characterised by what is stated in the characterising part of claim 13.

The use according to the present invention is characterised by what is stated in the characterising part of claim 22.

The apparatus according to the present invention is characterised by what is stated in the characterising part of claim 24.

The process according to the present invention can be used to produce etherified dimers from fresh feeds containing such olefinic hydrocarbon(s) which, when subjected to dimerisation reaction, form compounds that can be etherified. Such compounds are, for example, C₈-C₁₂ olefins. In theory, a compound can be etherified, if it contains at least one first carbon atom linked to a second carbon atom with a double bond, said first carbon atom having two other alkyl groups bonded to it. In practice, some limitations may arise if the olefin and/or the alcohol molecules are very bulky. Preferred fresh feed to the dimerisation comprises C₄-C₆-isoolefins, more preferably C₄ or C₅-isoolefins and in particular isobutene.

According to the invention, the olefinic hydrocarbon feed is contacted with an acidic catalyst in the presence of alcohol or other oxygenate in a dimerisation zone of a reaction system comprising at least one dimerisation zone, at least one distillation zone and at least one etherification zone. The conditions in said dimerisation zone are such that at least a part of the olefins in the feed is dimerised. The flow from said dimerisation zone is introduced into a distillation zone, where the main part of the dimerised reaction product is separated. A flow comprising alcohol or other oxygenate is circulated from the distillation zone back to the dimerisation. With the help of the recycling flow the conversion of the olefin(s) and the production of dimerised product is increased. The dimerised olefin is then etherified with alcohol to corresponding ether.

Considerable advantages are achieved by means of the present invention. The process is much faster than those suggested in prior art. The use of separate reaction zone and fractionation zone enables the use of different reaction temperatures and pressures. The most preferable reaction temperatures and pressures are not the same as the most preferable fractionation temperatures. Further, the reaction temperature can be adjusted so that it is at first high, thus making the reaction faster, and, subsequently, a low reaction temperature is used in order to increase the conversion. The catalyst can be easily removed and replaced in the reactor(s) in the reaction zone.

When using the process of the present invention, isoolefins can be converted to their dimers almost completely, and, thus, the feed to the etherification zone can consist essentially of desired dimers. In all, the dimerisation process is more selective than those of the prior art, which then increases the efficiency of the present process. The rate of the dimerisation reaction can be increased by increasing the temperature. This is especially preferred when tert-butyl alcohol (TBA) is used as the oxygenate.

The amount of the flow fed to the etherification zone can be optimised to match different production objectives. Thus, a part of the dimerised product can be fed to hydrogenation, while a part is first fed to etherification.

The octane number of the etherified dimer is high. If diisobutene is etherified with methanol, the blending octane number (BRON) of the obtained product is, according to the literature, 147 (Papachristos, M. J. et al., J. Inst. Energy 64 (1991) 113-123). It is, thus, extremely suitable for use as a fuel component. Due to the bulkiness of the etherified dimer molecule it is insoluble to water.

### BRIEF DESCRIPTION OF THE DRAWINGS

FIG. 1 depicts in a schematic fashion the process configuration of the basic technical solution of the invention.

FIG. 2 depicts the etherification zone arranged according to one embodiment of the present invention.

FIG. 3 depicts an embodiment in which the etherification zone comprises a side-reactor.

### DETAILED DESCRIPTION OF THE INVENTION

Definitions

For the purposes of the present invention, “distillation zone” designates a distillation system comprising one or more distillation columns. The distillation columns are operated so as to provide an overhead product and a bottoms product having different compositions. The columns are preferably connected in series. The feed plate can be selected for each column to be most advantageous in view of the overall process. Likewise, the plates for side-draw of flows to be recovered or circulated can be selected individually for each column. The distillation column can be any column suitable for distillation, such as a packed column, or one provided with valve, sieve or bubble-cap trays. In case of packed column the term plate means a horizontal position in a packed section.

A “dimerisation zone” comprises at least one, typically two or three, reactor(s) operated at conditions at which the olefins contained on the feed are dimerised. The reactor can be, e.g., a tubular reactor with multiple pipes, wherein the pipes are filled with catalyst. Other possibilities include a simple tubular reactor, a boiler reactor, a packed bed reactor and a fluidised bed reactor. The reactor used is preferably such in which the catalyst is placed in more than one layer and cooling is introduced between the layers. Preferably at least one of the reactors has a cooling system. For example, the pipes of the tubular reactor with multiple pipes can be cooled. Another example of a suitable reactor is a combination of a fixed bed reactor and a cooler, in which part of the reactor effluent can be circulated back to the reactor via the cooler. The operating pressure of the reactors depends on the type of the reactor and on the composition of the feed, typically it is desired to keep the reaction mixture in liquid phase.

An “etherification zone” is the zone of the process in which C₈-C₁₀ olefin, preferably the dimerised reaction product, is reacted with an alcohol or a mixture of alcohols at conditions at which a corresponding alkyl ether is formed. The etherification can be carried out using widely different configurations, which will be described in more detail below.

“Oxygenate” designates a compound containing oxygen. Typically, the oxygenates used in the present invention are primary, secondary or tertiary alcohols or water.

“Isooctene” and “diisobutene” are both products of isobutene dimerisation. Thus they can be used interchangeably to designate 2,4,4-trimethyl-1-pentene and 2,4,4-trimethyl-2-pentene or a mixture thereof.

“Dimer mixture” contains the product of the dimerisation reaction in the dimerisation zone. When, e.g., only C₄-olefins or only C₅-olefins are fed to the process, it is clear that the resulting product of the mutual reactions of the olefins yield dimers. However, when different olefins are present in the feed, also reactions between the olefins yielding, e.g., C₉-olefins, can occur in addition to dimerisation. For reasons of simpilicity, the word “dimer”, “dimerised olefins” or “dimerised reaction product” are in the following, therefore, to be understood to comprise also these reaction products.

The Etherification

According to the present invention, ether is produced in an etherification zone which comprises at least one reaction zone and at least one fractionation zone.

The flow containing C₈-C₁₂ olefin or a mixture of C₈-C₁₂ olefins is fed to a first reaction zone. Also alcohol or a mixture of alcohols is fed to the first reaction zone. The olefin(s) and the alcohol(s) are reacted in the presence of a catalyst at conditions at which corresponding alkyl ether(s) is formed. The effluent of the first reaction zone is conducted to a fractionation zone operated under conditions sufficient to produce an overhead flow and a bottoms product having different hydrocarbon compositions. From the fractionation zone is withdrawn a side-flow. The side-flow is conducted either back to the first reaction zone or to a second reaction zone. The effluent of the first or the second reaction zone is then fed back to the fractionation zone. The ether is recovered from the fractionation zone.

The reaction zone comprises at least one, preferably two or three reactors operated at conditions at which the olefin(s) react with alcohol(s) forming corresponding ether(s). The reactor(s) are selected independently from each other and are typically fixed or fluidised bed or tubular reactors, or essentially similar to those used in dimerisation (cf. above). When the reaction zone contains more than one reactor, they are preferably arranged in series (in a cascade), but they can also be arranged in parallel. If there are more than two reactors they may also be arranged in series/parallel. Preferably, the reaction mixture is cooled between the reactors, which is why heat-exchangers are preferably employed in the reaction zone(s).

The fractionation zone is operated under conditions sufficient for providing an overhead product and a bottoms product having different hydrocarbon compositions. In particular, the fractionation zone is operated so as to provide a different hydrocarbon composition in each stage of the zone. The fractionation zone typically comprises at least one distillation column. The distillation columns are typically essentially similar to those used in distillation zone (cf. above).

The olefin feed consists essentially of C₈-C₁₂ olefins, typically C₈-C₁₀ olefins, preferably C₈-C₁₀ isoolefins and in particular C₈ isoolefins. With isoolefins is meant olefins that contain at least one first carbon atom linked to a second carbon atom with a double bond, said first carbon atom having two other alkyl groups bonded to it. According to a preferred embodiment of the present invention, the feed comprises at least 85 wt-% of said olefins. Typically, the feed comprises 85-100 wt-%, in particular 85-95 wt-% of a mixture of 2,4,4-trimethyl-1-pentene and 2,4,4-trimethyl-2-pentene.

The alcohol feed to the etherification zone can be combined with the C₈-C₁₂ olefin feed, but it is also possible to feed it directly to any or all of the reaction zone(s) of the etherification zone.

The alcohol(s) used in the etherification are typically primary or secondary alcohols, preferably C₁-C₆ alcohols, more preferably methanol or ethanol, and in particular methanol. Additional alcohol can optionally be fed to any or all of the reactor(s) in any or all reaction zone(s).

An acidic catalyst is used in the etherification zone. Suitable catalysts include, for example, ion-exchange resins. As catalysts can, however, be used zeolites and other inorganic catalysts. The ion-exchange resin typically used can comprise sulphonic acid groups and it can be prepared by polymerising or copolymerising aromatic vinyl compounds and, thereafter, sulphonating. As examples of aromatic vinyl compounds the following may be mentioned: styrene, vinyl toluene, vinyl naphthalene, vinyl ethyl benzene, methyl styrene, vinyl chlorobenzene, and vinyl xylene. An acidic ion-exchange resin contains typically sulphonic acid groups 4.5 . . . 5.5 eq/kg. Preferred resins are those based on copolymers of aromatic monovinyl compounds and aromatic polyvinyl, in particular divinyl, compounds, in which the concentration of polyvinylbenzene is approximately 1 . . . 20 wt-% of the copolymer. The particle size of the ion-exchange resin is preferably approximately 0.15 . . . 1 mm.

In addition to the resins already described, also perfluorosulphonic acid resins consisting of copolymers of sulphonylfluorovinyl ethyl and fluorocarbon compounds can be used.

Various suitable ion-exchange resins are commercially available; an example of these is Amberlyst 15.

According to one embodiment of the present invention the catalyst used in the etherification zone of the present process is based on a polyolefin backbone to which a compound which contains reactive groups is grafted, where after a functional group is introduced into the fibrous structure. The compound which contains reactive groups is preferably styrene, and the functional group is preferably an acid group, in particular a sulfonic acid group.

The temperature in the reaction zone(s) of the etherification zone is typically approximately from 40 to 120° C., preferably from 50 to 105° C. The pressure in the reactors is typically 20 bar or less, often just a slight overpressure is used to keep the mixture in liquid phase.

The effluent from the reaction zone(s) is fed to the fractionation zone. The feed to the fractionation zone is typically arranged to a stage between the top and the bottom of the zone, preferably to the stage that makes the separation easiest. The side-flow is typically withdrawn from a stage between the feed point and the top of the fractionating zone. The pressure of the side-flow is optionally increased, for example, by means of a pump. The temperature of the side-flow can be increased or decreased with a heat-exchanger to the most optimal etherification reaction temperature.

Typically, the fractionation zone comprises at least one distillation column. The temperature at the top of the distillation column is about 40 to 70° C., typically about 50 to 60° C., and at the bottom of the column about 110 to 170° C., typically about 150 to 160° C. The pressure in the distillation column is preferably in the range of 1 to 10 bar. The temperatures and pressures are in practice selected according to the availability of local heating and cooling media.

The overhead product of the distillation column typically comprises light hydrocarbons. The desired etherified reaction product is recovered from the fractionation zone, typically as the bottoms product of the fractionation zone (or column). According to a preferred embodiment, the feed to the etherification zone consists mainly (preferably at least 50 wt-%) of 2,4,4-trimethyl-1-pentene and 2,4,4,-trimethyl-2-pentene. Methanol is used as the alcohol in the etherification reaction, and, thus, 2-metoxy-2,4,4-trimethyl pentane is obtained as the product. According to literature (Papachristos, M. J. et al., J. Inst. Energy 64 (1991) 113-123), the blending octane number (BRON) of the obtained ether is high (about 147).

An example of an etherification zone arranged according to a first preferred embodiment of the present invention is disclosed in detail in FIG. 2. The reaction zone comprises at least one, typically two or three, reactor(s) 21, 22, 23 preferably arranged in series (in a cascade). Due to the exothermic etherification reaction the temperature rises in each reactor from 5 to about 35° C. The reaction mixture is preferably cooled between the reactors, and, thus, a heat-exchanger 24, 25 is preferably arranged between the reactors. The effluent of the last reactor in series is fed to the fractionation zone 26. Typically, the fractionation zone consists essentially of one distillation column.

The bottoms product B1 of the fractionation zone 26 comprises the alkyl ether. The unreacted reagents, namely the unreacted C₈-C₁₂ olefin(s) and the alcohol(s), which typically form an azeotrope, are recycled to the reactor(s), preferably to the first reactor in the series, as recycling flow R1. The recycling flow R1 is preferably taken from a plate above the feed plate of the distillation column 26. The overhead product D1 of the fractionation zone comprises light components and possibly even alcohol, although preferably the conditions in the fractionation zone are optimised so that no alcohol is present in the overhead.

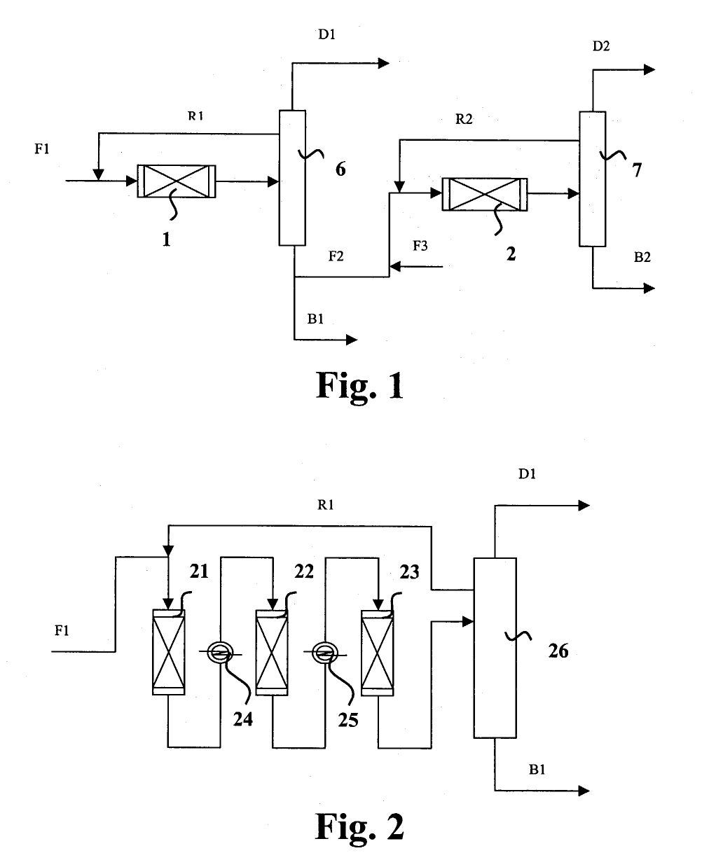

An alternative description of the preferred embodiment is presented in FIG. 1, where the side-flow R2 containing unreacted olefin(s) and alcohol(s) is withdrawn from the fractionation zone 7 and returned to the first reaction zone 2.

According to this embodiment, C₈-C₁₂ olefin feed is fed to a first reaction zone 2. The effluent of the first reaction zone is fed to the fractionation zone 7. The side-flow R2 containing unreacted olefin(s) and alcohol(s) is withdrawn from the fractionation zone 7 and returned to the first reaction zone 2. In the figure, side-flow R2 is combined with the C₈-C₁₂ olefin feed F2, but it could as well be fed directly to the first reaction zone 2 or be combined with methanol feed F3. Likewise, the methanol feed F3 is, according to FIG. 1, combined with the olefin feed F2, but it could as well be fed directly to the first reaction zone 2. The ether is recovered as the bottoms product B2 of the fractionation zone. The overhead D2 of the fractionation zone 7 contains light hydrocarbons.

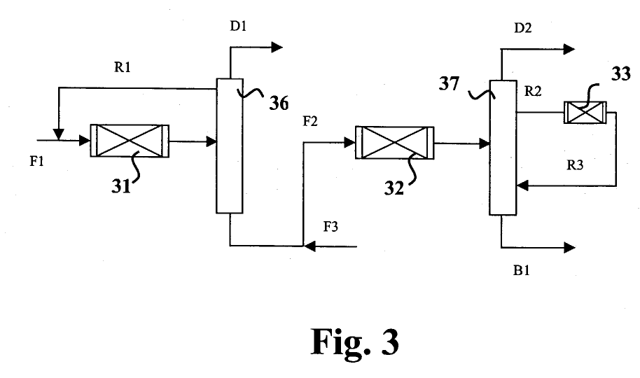

According to a second preferred embodiment of the present invention, one example of which is presented in FIG. 3, the side-flow containing unreacted olefin(s) and alcohol(s) from the fractionation zone 37 is fed to a second reaction zone 33.

According to this embodiment, the C₈-C₁₂ olefin feed F2 together with alcohol feed F3 is conducted to a first reaction zone 32. The effluent of the first reaction zone 32 is fed to a fractionation zone 37. From the fractionation zone 37 is withdrawn a side-flow R2, which typically contains unreacted olefins and alcohol. The side-flow R2 is fed to a second reaction zone 33, where the alcohol(s) and olefin(s) present in the side-flow R2 are subjected to an etherification reaction in the presence of a catalyst. The second reaction zone 33 contains one, two, three or more reactor(s) preferably connected in series (in a cascade). Heat-exchangers are then preferably employed between the reactors to lower the temperature of the reaction mixture. Optionally, additional alcohol is fed to the second reaction zone 33 to any or all of the reactors (not shown). The effluent R3 of the second reaction zone 33 is then returned to the fractionation zone 37. The effluent R3 is typically returned to the fractionation zone 33 to a stage lower than the draw-off of the side-flow, preferably to a stage lower than the feed stage of the fractionation zone.

Ether is recovered as the bottoms product B1 of the fractionation zone 37, and the overhead product D2 of the fractionation zone 37 typically comprises light hydrocarbons and, possibly, minor amounts of alcohol.

According to one embodiment of the present invention, the etherification is carried out by conventional reactive distillation. In said system the etherification reaction and the separation of the products takes place at least partly simultaneously. Thus, the C₈-C₁₂ olefin(s) and alcohol or a mixture of two or more alcohols are either together (preferred) or separately fed into a fractionation zone which comprises catalyst placed inside the fractionation zone. In particular, the fractionation zone is operated so as to provide a different hydrocarbon composition in each stage of the zone.

With regard to the reactive distillation, reference is made to the embodiments described in greater detail in International Patent Application WO 93/19032.

The Overall Process

In the following is described the process which is preferably used for producing C₈-C₁₂ olefins used as the feedstock for the etherification reaction. These two processes are advantageously combined to be able to produce ether suitable for use as a fuel component from olefinic hydrocarbon(s).

According to the invention, the hydrocarbon fresh feed containing dimerisable olefin(s) is contacted with a catalyst together with alcohol or other oxygenate in a dimerisation zone at conditions at which at least a part of the olefins is dimerised. In case where the olefin feed comprises, for example, both C₄- and C₅-olefins, also reactions between different olefins occur, thus forming C₉-olefins. In addition also small amounts of other oligomers, such as trimers or tetramers are formed in the reaction. The flow from the dimerisation zone is introduced into a first distillation zone, where at least the main part of the dimerised reaction product is separated from unreacted olefins and oxygenate.

A flow comprising alcohol, other oxygenate and/or the product(s) of the reaction between the oxygenate and the olefin(s) is withdrawn from the distillation zone and returned to the dimerisation zone. With the help of the recycling the conversion of the olefin and the production of dimerised product is increased. It is to be understood that although the following description refers to a side-flow in singular, which is the typical configuration, it is also possible to withdraw two or more side-flows containing oxygenate and circulate all those flows back to dimerisation.

From the distillation zone is obtained a flow which comprises the dimerised olefin(s). This flow is conducted to an etherification zone. Fresh alcohol is also fed to the etherification zone.

According to a preferred embodiment, an example of which is disclosed in FIGS. 1 and 3, the distillation zone 6; 36 comprises at least one distillation column, and the flow R1 comprising the oxygenate is withdrawn from the side of the column 6; 36.

According to another preferred embodiment the effluent of the dimerisation zone is conducted to a distillation zone which comprises at least one distillation column. From the side of the distillation column, preferably from a point between the feed point of the column and the top of the column, is withdrawn a flow comprising unreacted olefins and oxygenate. This flow is conducted to a second dimerisation zone comprising at least one reactor. The effluent of the second dimerisation zone is conducted to the distillation column, preferably to a point below the feed point of the distillation column and below the point where the side-flow is withdrawn. The overhead of the distillation column, which typically comprises some amounts of oxygenate, is returned to the first dimerisation zone, and from the side of the distillation column, typically from a point between the top of the column and the point where the side-flow is withdrawn, a flow comprising light hydrocarbons is withdrawn and conducted to, e.g., further processing. The dimerised reaction product is obtained as the bottoms product of the distillation zone.

The fresh feed (i.e., the fresh olefin(s) fed to the dimerisation zone) of the present process comprises olefinic hydrocarbon(s). In other words, the fresh feed comprises at least one olefin capable of dimerising so that the formed dimer has two alkyl groups attached to a carbon atom which is bound with a double bond to another carbon atom. The feed comprises dimerisable olefins at least 5 wt-%, preferably at least approximately 20 wt-%. The dimerisable olefins(s) in the feed are typically C₄-C₆-olefins, preferably C₄-C₅-isoolefins and in particular isobutene.

Preferably, the feed comprises a fraction obtained from isobutane dehydrogenation, when the feed comprises mainly isobutene and isobutane and possibly small amounts of C₃- and C₅-hydrocarbons. Typically the feed then comprises 40-60 wt-% of isobutene and 60-40 wt-% isobutane, usually there is 5-20% less isobutene present than isobutane. Thus, the ratio of isobutene to isobutane is approximately 4:6 . . . 5:5.5. As an example of an isobutane dehydrogenation fraction, the following can be presented: 45 wt-% isobutene, 50 wt-% isobutane and other inert C₄-hydrocarbons and approximately 5 wt-% of C₃-, C₅- and heavier hydrocarbons altogether.

The following feedstock can also be used: Fluid Catalytic Cracking (FCC) gasoline, light FCC gasoline, Thermal Catalytic Cracking (TCC) gasoline, DCC and Residual Catalytic Cracking (RCC) gasoline, pyrolysis-C₅-gasoline and Coker gasoline, and the C₄-fraction after the removal of butadiene, also called Raffinate 1 of an ethylene or FCC unit. In practice, a fraction readily available comprises C₄ and/or C₅ fractions from FCC.

According to a preferred embodiment, the feed comprises mainly isobutene, and typically at least 5 wt-%, preferably at least 20 wt-% of the total; mass flow of the olefinic hydrocarbons present in the feed is isobutene. Naturally, the feed can consist of pure isobutene, but in practice, the feedstock readily available comprises C₄-based hydrocarbon fractions from oil refining.

Raffinate 1 obtained from ethylene cracking is typically composed of approximately 50 wt-% isobutene, approximately 25 wt-% linear butenes and approximately 25 wt-% paraffins. The C₄ fraction obtained from the FCC is typically composed of 10-50, in particular 10-30 wt-% isobutene, 20-70 wt-% 1- and 2-butene and approximately 5-40 wt-% butane. As an example of a typical FCC-mixture, the following can be presented: approximately 30 wt-% isobutene, approximately 17 wt-% 1-butene, approximately 33 wt-% 2-butene and approximately 20 wt-% butane.

Also isobutene prepared from chemicals can be used as fresh feed.

C₅-olefins present in the fresh feed include, for example, linear pentene, 2-methyl-1-butene, 2-methyl-2-butene, 3-methyl-1-butene or 2-ethylpropene.

In addition to the olefinic hydrocarbon(s), an oxygenate or a mixture of two or more oxygenates is fed into the dimerisation zone of the process in order to slow down the oligomerisation reactions of the olefin and to decrease the catalyst deactivation. The oxygen-containing compound can be fed together with the fresh olefin feed, or it can be fed together with the circulation flow, or directly to the reaction zone.

The oxygenate protects the catalyst by hindering the formation of large molecules, since the heavier components forming from trimers and tetramers block the catalyst. The use of oxygenate increases the dimer selectivity whereby the portion of trimers and tetramers of the olefin oligomers decreases.

According to the present invention, water, ether or alcohol, preferably C₁-C₆ alcohol (e.g. methanol, ethanol, isopropanol or t-butanol) is used as the oxygenate. As obvious from the list, the alcohol can be primary, secondary or tertiary alcohol. Further examples include tert-amyl methyl ether, 2-butanol and 2-pentanol.

Oxygenates can be divided into two classes: those which react with the olefins present in the fresh feed and those which are inert at the dimerisation conditions.

To the first class belong primary alcohols, such as ethanol, methanol and propanol and water. Water reacts with isoolefin(s) and forms tertiary alcohol, for example, tert-butyl alcohol, TBA in the reaction between water and isobutene or tert-amyl alcohol in the reaction between water and 2-methyl-1-butene or 2-methyl-2-butene. The reaction between water and linear olefin(s) produces secondary alcohols. Thus, for example the reaction between water and 2-butene results in sec-butyl alcohol. When the feed comprises different olefins, also mixtures of the above described alcohols are obtained.

The hydrocarbon feedstock obtained from one of the oil refining unit operations described above usually contain water 50-500 ppm, in particular 100-300 ppm. In some cases, the water present in the fresh hydrocarbon feedstock is enough to protect the catalyst and thus there is no need to feed additional oxygenate to the process. This is particularly true when the C₄-olefin content in the feed is less than 10 wt-%, in particular less than 5 wt-%.

Preferably, however, an oxygenate is used, which does not significantly react with the olefins, such as TBA.

The molar ratio of oxygenate and olefin, e.g., TBA and isobutene, in the fresh feed is smaller than the stoichiometric ratio, preferably the ratio is kept below 0.2. It is important to adjust the amount of oxygenate to the feedstock used. The amount of oxygenate needed in the dimerisation of C₅-olefins is small, typically its content in the reaction zone is in the range of 50-500 ppm, in particular 100-300 ppm. When the fresh feed contains both C₄- and C₅-olefins, typically the amount of oxygenate needed increases as the fraction of C₄-olefins increases.

An acidic catalyst is used in the dimerisation zone. Suitable catalysts include, for example, ion-exchange resins. As catalysts can, however, be used zeolites and other inorganic catalysts. Thus, the ion-exchange resin typically used can comprise sulphonic acid groups and it can be prepared by polymerising or copolymerising aromatic vinyl compounds and, thereafter, sulphonating. As examples of aromatic vinyl compounds the following may be mentioned: styrene, vinyl toluene, vinyl naphthalene, vinyl ethyl benzene, methyl styrene, vinyl chlorobenzene, and vinyl xylene. An acidic ion-exchange resin contains typically sulphonic acid groups 4.5 . . . 5.5 eq/kg. Preferred resins are those based on copolymers of aromatic monovinyl compounds and aromatic polyvinyl, in particular divinyl, compounds, in which the concentration of polyvinylbenzene is approximately 1 . . . 20 wt-% of the copolymer. The particle size of the ion-exchange resin is preferably approximately 0.15 . . . 1 mm.

In addition to the resins already described, also perfluorosulphonic acid resins consisting of copolymers of sulphonylfluorovinyl ethyl and fluorocarbon compounds can be used.

Various suitable ion-exchange resins are commercially available, an example of these is Amberlyst 15.

The concentration of the catalyst is typically 0.01-20%, preferably approximately 0.1-10% of the weight of the liquid mixture to be handled.

The temperature in the dimerisation zone is typically 50-120° C. The upper level of the temperature range is set by the heat-resistance properties of the catalyst. The reaction can very well be carried out at temperatures higher than 120° C., for example up to 160° C. or even higher. The formation of the dimers can be enhanced by increasing the temperature during the reaction. On the other hand, a lower temperature favours the formation of ether.

The flow from the dimerisation zone is conducted to a distillation zone, where components are separated from one another. From the distillation zone, a flow comprising alcohol or ether or the mixture thereof is withdrawn. When using alcohol which does not significantly react with the olefin (such as TBA), the recycling flow comprises the major part of the alcohol present in the reactor effluent. When using alcohol which does react with the olefin (such as methanol with isobutene), the side-draw can comprise both alcohol and ether.

The flow from the distillation zone is typically withdrawn from a distillation column from a plate higher than the feed plate. The side-draw is circulated back to dimerisation. The amount of the circulated flow can be altered as well as the point to which it is conducted (for example, either directly to the reaction zone or to the fresh feed). The mass flow of the circulated flow is typically 0.01 . . . 10 times, preferably 1 . . . 5 times the mass flow of fresh hydrocarbon feed.

The oxygenates form readily azeotropes with the olefins present in the feed or formed in the dimerisation. For example, TBA forms an azeotrope with isooctene. The azeotropes can be decomposed by the addition of another compound, which forms an azeotrope with the oxygenate more readily than the olefin. The azeotrope-breaking compound can also be present in the feed originally, and thus no special feed is required. In this case, the azeotrope-breaking compound only has to be kept in the circulation and not taken out from the reaction system. A good example of this kind of compound are C₆-hydrocarbons, which break the TBA-isooctene azeotrope described above, thus enabling the recovery of the desired dimer. As discussed, C₆-olefins are typically present in the C₅-fraction of FCC.

The dimerised reaction product is obtained as the bottoms product from the first distillation zone. The product flow typically contains olefin oligomers (dimers and trimers). The fraction of dimers of the formed olefin oligomers is typically at least 80 wt-%. When isobutene is used as the dimerised olefin, the weight ratio of dimers to trimers in the bottoms product is, e.g., 99:1.80:20. Most of the dimers obtained are 2,4,4-trimethyl-pentenes, i.e., the double bond is in a carbon atom to which a-methyl group is attached. Typically, at least 20 wt-%, preferably at least 50 wt-% and in particular at least 65 wt-% of the dimerised reaction product consists of 2,4,4-trimethyl pentenes, i.e. 2,4,4-trimethyl-1-pentene and 2,4,4-trimethyl-2-pentene. The fraction of other trimethyl pentenes as well as the fraction of dimethyl hexenes in the mixture remains extremely small.

When C₅ olefins are dimerised, various C₁₀ isomers are formed. Those C₁₀ olefins which have the double bond in a carbon atom to which also two alkyl groups are attached, can be further etherified. It is to be noted, however, that as the molecule is relatively bulky, the rate of etherification is slow. Same applies for the C₉ olefins produced in the reaction between C₄ and C₅ olefins.

The distillation zone is optionally operated so that only dimers are incorporated to the flow which is fed to the etherification. The bottoms product of the distillation zone typically contains also trimers and/or small amounts of oxygenate. Typically, the fraction of dimerised olefins (i.e., C₈-C₁₂ olefins) in the bottoms product of the distillation zone is 65-100 wt-%, in particular 85-95 wt-%, preferably at least 85 wt-%, and in particular at least 90 wt-%. The bottoms product of the distillation zone can thus be used as the feed to the etherification zone as such.

According to one embodiment of the present invention, a fractionating unit is added between the first distillation zone and the etherification zone. By-means of the fractionating unit it is possible to adjust the composition of the feed to etherification to match each situation.

According to another preferred embodiment of the present invention, only a part, e.g. approximately 5-80 wt-%, preferably 10-50 wt-%. and in particular 20-75 wt-% of the dimerised product is subjected to etherification. The rest of the dimerised product can be, for example, hydrogenated to form a fuel component, which also has a high octane number. The octane number of the ethers obtained by the present process is extremely high, and, therefore, even relatively small amounts of the ether product increase the octane number of the fuel.

Advantageously, the overall etherification process is carried out in an apparatus comprising in combination (cf. FIG. 1)

at least one first reactor 1 having an inlet for feeding olefinic hydrocarbon feedstock, and an outlet for the withdrawal of a first reaction mixture,

at least one first distillation column 6 having at least one inlet connected to the outlet of said reactor 1 for fractionating the first reaction mixture withdrawn from the first reactor 1, said distillation column 6 having a bottoms outlet and at least one outlet for the withdrawal of a side-flow, said outlet of the side-flow being connected to the inlet of the first reactor 1,

at least one second reactor 2 having at least one inlet connected to the bottoms outlet of the first distillation column 6, said at least one second reactor 2 having an outlet for the withdrawal of the second reaction mixture, and

at least one second distillation column 7 having an inlet connected to the outlet of the second reactor 2 for fractionating the second reaction mixture, said second distillation column having a bottoms outlet and at least one outlet for the withdrawal of a side-flow, said side-flow being connected to the inlet of the second reactor 2.

According to the second preferred embodiment of the present invention (cf. FIG. 3), the apparatus further comprises a third reactor 33 having an inlet for feedstock and an outlet for the withdrawal of a third reaction mixture. According to the this embodiment, the outlet for the withdrawal of the side-flow of the second distillation column 37 is then connected to the inlet of the third reactor 33 in addition to or instead of being connected to the inlet of the second reactor 32. The outlet of the third reactor 33 is then connected to an inlet of the second distillation column 37, said inlet being different from the inlet which is connected to the second reactor 32.

It is to be understood, that one or more additional reactor(s) may be employed after the first, second and third reactor. The reactors are then typically arranged in series. The feed to these reactors is the effluent of the previous reactor, and the effluent of the last reactor in series is conducted to the distillation zone. Optionally, heat-exchanger is arranged between each reactor.

The reactors are selected as indicated above. Optionally, the side-flow(s) is (are) subjected to cooling with the help of one or more heat-exchanger(s) prior to feeding to the reactors.

The invention is further illustrated with the aid of the following non-limiting examples.

### EXAMPLES

Two examples are presented in order to further illustrate the invention. Experimental kinetic studies form the basis for the first example. The indicated process configuration has then been simulated on the basis of the models obtained from the experimental results.

The total hydrocarbon feed (excluding methanol) is set to be 100 mol/h. A conversion of approximately 99% of trimethylpentenes has also been set as a criterion.

Example 2 is an experimental example without simulation.

### Example 1

The process configuration according to FIG. 2 was simulated for producing 2-metoxy-2,4,4-trimethyl pentane from an olefinic hydrocarbon feed containing 2,2,4-trimethyl-1-pentene (approx. 76 wt-%) and 2,2,4-trimethyl-2-pentene (approx. 24 wt-%). The molar ratio between the trimethylpentenes and methanol was 1.

In Table 1 is presented the molar flows and mass flows of the major components of each flow.

TABLE 1

| Flow | | F1 | R1 | F2 | F3 | F4 | D1 | B1 |
|---|---|---|---|---|---|---|---|---|
| Pressure | kPa | 800 | 100 | 1800 | 800 | 800 | 100 | 100 |
| Temp. | C | 79.85 | 58.21 | 83.41 | 61.12 | 38.37 | 57.67 | 143.43 |
| Mol. weight | g/mol | 72.13 | 96.66 | 95.21 | 99.20 | 100.87 | 53.51 | 144.23 |
| Molar flow | Total | 100.00 | 500.00 | 583.35 | 559.92 | 550.64 | 1.00 | 49.64 |
| mol/h | MeOH | 50.00 | 102.71 | 136.06 | 112.63 | 103.36 | 0.73 | 0.00 |
| | 224TMP | 0.00 | 0.00 | 0.00 | 0.00 | 0.00 | 0.00 | 0.00 |
| | 244TMP-1 | 38.00 | 292.10 | 321.79 | 300.62 | 292.56 | 0.19 | 0.02 |
| | 244TMP-2 | 12.00 | 90.89 | 94.55 | 92.30 | 91.08 | 0.08 | 0.02 |
| | TOME | 0.00 | 14.29 | 30.94 | 54.37 | 63.65 | 0.00 | 49.60 |
| Mass flow | Total | 7.21 | 48.33 | 55.54 | 55.54 | 55.54 | 0.05 | 7.16 |
| kg/h | MeOH | 1.60 | 3.29 | 4.36 | 3.61 | 3.31 | 0.02 | 0.00 |
| | 224TMP | 0.00 | 0.00 | 0.00 | 0.00 | 0.00 | 0.00 | 0.00 |
| | 244TMP-1 | 4.26 | 32.78 | 36.11 | 33.73 | 32.83 | 0.02 | 0.00 |
| | 244TMP-2 | 1.35 | 10.20 | 10.61 | 10.36 | 10.22 | 0.01 | 0.00 |
| | TOME | 0.00 | 2.06 | 4.46 | 7.84 | 9.18 | 0.00 | 7.16 |

As can be seen from the table, the bottoms product B1 consists essentially of the desired product, herein referred to as tert-octyl methyl ether (TOME). In practice, the distillate flow D1, and, thus, the amount of methanol leaving the distillation column, would be minimised, typically at least essentially all the unreacted methanol would then be incorporated in the return flow R1.

The olefin feed of this example consists of 2,4,4-trimethylpentenes. In practice, the dimerised reaction product is often obtained as the bottoms product of the first distillation zone, and the bottoms product typically contains some amount of other oligomers, i.e., trimers and tetramers of the olefins present in the feed. If these oligomers are not separated from the feed to the etherification zone, the oligomers would then be present in the bottoms product of the etherification zone as well.

### Example 2

The mixture of 2,4,4-trimethyl-1-pentene and 2,4,4-trimethyl-2-pentene (ratio of isomers 3.5:1) was etherified with methanol in a batch reactor. The initial ratio of methanol to alkene was 1:1 mol:mol. The catalyst was a commercial ion-exchange resin, Amberlyst 35 (Rohm & Haas). The concentration of the catalyst in the reaction mixture was 1.8-2.7 wt-%. The following yields of ether were achieved in 24 hours

| T/° C. | yield of ether | batch time/h |
|---|---|---|
| 50 | 6.9% | 24.1 |
| 60 | 11.1% | 24 |
| 70 | 12.6% | 24.2 |
| 80 | 9.9% | 24.8 |

or in 7 hours

| T/° C. | yield of ether | batch time/h |
|---|---|---|
| 50 | 2.4% | 6.9 |
| 60 | 5.7% | 6.8 |
| 70 | 8.7% | 7.8 |
| 80 | 7.9% | 7.5 |

In batch reactor the reaction is very slow. The batch time can be shortened e.g. by using more catalyst per batch or by exploiting temperature steps. The higher the temperature the faster the reaction but the lower the equilibrium. Thus it is advantageous to start the reaction at high temperature and lower the temperature as the reaction proceeds.

The same reaction was carried out at same conditions with a fibrous Smopex-101 catalyst (Smoptech Ltd.). Initially, the reaction was approximately four times faster than with Amberlyst ion-exchange resin.

The above presented experiments clearly reveal that a traditional batch reactor is not the optimal system for carrying out this reaction, but the more sophisticated methods introduced here are more suitable.

1. A process for producing ether, comprising
feeding a flow consisting essentially of C₈-C₁₂ olefin(s) to a first reaction zone (2; 21-25; 32) comprising at least one reactor,
feeding alcohol(s) capable of reacting with the olefin(s) to said first reaction zone (2; 21-25; 32),
subjecting the olefin(s) and alcohol to an etherification reaction in the presence of an acidic catalyst,
feeding the effluent from the first reaction zone (2; 21-25; 32) to a fractionation zone (7; 26; 37) operated under conditions sufficient to provide an overhead product and a bottoms product having different compositions,
withdrawing a side-flow from the fractionation zone (7; 26; 37),
conducting the side-flow either back to the first reaction zone (2; 21-25; 32) or to a second reaction zone (33),
circulating the effluent of the first reaction zone (2; 21-25; 32) or the second reaction zone (33) back to the fractionation zone (7; 26; 37), and
recovering the ether from the fractionation zone (7; 26; 37).

2. The process according to claim 1, wherein at least 85 wt-%, preferably at least 90 wt-% of the feed consists of C₈-C₁₂ olefins.

3. The process according to claim 1 or 2, wherein the feed comprises C₈-C₁₀ isoolefins, preferably C₈ isoolefins.

4. The process according to any of claims 1-3, wherein at least 20 wt-%, preferably at least 50 wt-% and in particular at least 85 wt-% of the feed consists of a mixture of 2,4,4-trimethyl-1-pentene and 2,4,4-trimethyl-2-pentene.

5. The process according to any of the preceding claims, wherein the alcohol is a primary or secondary C₁-C₆-alcohol, preferably methanol.

6. The process according to any of the preceding claims, wherein the catalyst is an acidic ion-exchange resin.

7. The process according to any of claims 1-5, wherein the catalyst is based on a polyolefin backbone, to which a compound that contains reactive groups is grafted, where after a functional group is introduced into the fibrous structure.

8. The process according to any of the preceding claims, wherein
the effluent of the first reaction zone (2; 21-25; 32) is fed to the fractionation zone (7; 26; 37) to a feed stage,
the fractionation zone (7; 26; 37) is operated so as to provide a different hydrocarbon composition in each stage of the zone, and
a side-flow is withdrawn from a stage different than the feed stage.

9. The process according to claim 8, wherein the side-flow is taken from a stage above the feed stage of the effluent of the first reaction zone (2; 21-25; 37).

10. The process according to any of the preceding claims, wherein fresh alcohol is fed to the first reaction zone (2; 21-25; 37) or to the second reaction zone (33).

11. The process according to any of the preceding claims, wherein the effluent of the first reaction zone (2; 21-25; 37) or second reaction zone (33) is returned to the fractionation zone (7; 26; 37) below the place from where the side-flow was withdrawn.

12. The process according to any of the preceding claims, wherein the etherified product is recovered as the bottoms product of the fractionation zone (7; 26; 37).

13. A process for etherification of olefins, comprising
feeding a flow comprising olefinic hydrocarbon(s) to a system including at least one dimerisation zone (1; 31), at least one distillation zone (6; 36), and at least one etherification zone, said etherification zone comprising at least one reaction zone (2; 32, 33) and at least one fractionation zone (7; 37),
contacting said flow with an acidic catalyst in the presence of an oxygenate at conditions at which at least a part of the olefin(s) dimerises,
feeding the effluent from said dimerisation zone (1; 31) to said distillation zone (6; 36) where the dimerised reaction product is separated from said effluent,
circulating a flow comprising oxygenate from the distillation zone (6; 36) back to the dimerisation zone (1; 31),
conducting at least part of the dimerised reaction product to the etherification zone (2, 7; 32-33, 37),
feeding alcohol to the etherification zone (2, 7; 32-33, 37),
subjecting the dimerised reaction product and alcohol to an etherification reaction in the presence of an acidic catalyst in a first reaction zone (2; 32) of the etherification zone,
feeding the effluent from the first reaction zone (2; 32) to a fractionation zone (7; 26; 37) operated under conditions sufficient for providing an overhead product and bottoms product having different hydrocarbon compositions,
withdrawing a side-flow from the fractionation zone (7; 37),
conducting the side-flow either to the first reaction zone (2; 32) or to a second reaction zone (33),
circulating the effluent of the first reaction zone (2; 32) or the second reaction zone (33) back to the fractionation zone (7; 37), and
recovering the ether from the fractionation zone (7; 37).

14. The process according to claim 13, wherein the olefinic hydrocarbon(s) is (are) fed to the dimerisation zone (1; 31) of the process.

15. The process according to claim 13 or 14, wherein the olefinic hydrocarbon(s) comprise at least 5 wt-%, preferably at least 20 wt-% isobutene.

16. The process according to any of claims 13-15, wherein the catalyst used in the dimerisation zone (1; 31) is an acidic ion-exchange resin.

17. The process according to any of the claims 13-16, wherein the oxygenate used in the dimerisation zone (1; 31) does not react with the olefin(s) contained in the feed.

18. The process according to claim 17, wherein the oxygenate is a tertiary alcohol having 4-6 carbon atoms, preferably tert-butyl alcohol.

19. The process according to any of claims 13-18, wherein the dimerised reaction product is obtained as the bottoms product of the distillation zone (6; 36).

20. The process according to any of claims 13-19, wherein the at least 20 wt-% of the dimerised reaction product consists essentially of 2,4,4-trimethyl-1-pentene and 2,4,4-trimethyl-2-pentene.

21. The process according to any of claims 13-20, wherein 5-100 wt-%, preferably 10-80 wt-%, and in particular 20-75 wt-% of the dimerised reaction product is conducted to the etherification zone.

22. The use of the dimerised reaction product obtained from a process which comprises the steps of
feeding a flow comprising olefinic hydrocarbon(s) to a system including at least one dimerisation zone (1; 31) and at least one distillation zone (6; 36),
contacting said flow with an acidic catalyst in the presence of an oxygenate at conditions at which at least a part of the olefin(s) dimerises,
feeding the effluent from said dimerisation zone (1; 31) to said distillation zone (6; 36) where the dimerised reaction product is separated from said effluent,
circulating a flow comprising oxygenate from the distillation zone (6; 36) back to the dimerisation zone (1; 31), and
recovering the dimerised reaction product from the distillation zone, as the olefinic feedstock in an etherification process.

23. The use according to claim 22, wherein the olefinic hydrocarbon(s) comprise at least 20 wt-% of isobutene.

24. An apparatus for producing an etherification product, comprising in combination:
at least one dimerisation reactor (1) having an inlet nozzle for a feed of a hydrocarbon feedstock and an outlet nozzle for withdrawal of a dimerization product;
at least one first distillation column (6) connected to the outlet nozzle of the dimerisation reactor (1) for fractionating the dimerisation product withdrawn from the dimerisation reactor (1) and having a bottoms outlet nozzle and an outlet nozzle for a sidedraw, said outlet nozzle of the sidedraw being connected to the inlet nozzle of the dimerization reactor (1);
at least one etherification reactor (2) having an inlet nozzle connected to the bottoms outlet of the first distillation column (6) and further having an outlet nozzle for withdrawal of an etherification product; and
a second distillation column (7) connected to the outlet nozzle of the etherification reactor (2) for fractionating the etherification product and having a bottoms outlet nozzle and an outlet nozzle for a sidedraw, said outlet nozzle of the sidedraw being connected to the inlet nozzle of the etherification reactor (2).
